(19) United States
(12) Patent Application Publication
(10) Pub. No.: US 2022/0026424 A1
(43) Pub. Date: Jan. 27, 2022

(54) MICROSTRUCTURE, METHOD FOR MANUFACTURING SAME, AND MOLECULE DETECTION METHOD USING SAME

(71) Applicant: NATIONAL INSTITUTE OF ADVANCED INDUSTRIAL SCIENCE AND TECHNOLOGY, Tokyo (JP)

(72) Inventors: Hyonchol KIM, Tsukuba-shi (JP); Dai KATO, Tsukuba-shi (JP); Naoshi KOJIMA, Tsukuba-shi (JP); Shohei YAMAMURA, Takamatsu-shi (JP); Tomoyuki KAMATA, Tsukuba-shi (JP)

(21) Appl. No.: 17/431,128

(22) PCT Filed: Feb. 26, 2020
(86) PCT No.: PCT/JP2020/007641

§ 371 Date: Aug. 13, 2021

(30) Foreign Application Priority Data
Feb. 27, 2019 (JP) 2019-034275

(51) Int. Cl. G01N 33/543; G01N 21/66; G01N 33/574; G01N 33/553

(52) U.S. Cl. CPC G01N 33/54346; G01N 21/66; B82Y 35/00; G01N 33/54326; G01N 33/553; G01N 33/574

(57) ABSTRACT

In order to provide a specific solution for producing a microstructure equipped with a mechanism for selectively detecting a marker molecule expressed by a target cell, or a specific biomolecule, and for detecting and identifying a molecule to be detected using the microstructure, the present invention provides a nearly hemispherical shell-shaped structure made of a first conductive material, and an electrode layer made of a second conductive material disposed on the concave side of the nearly hemispherical shell-shaped structure, wherein the first conductive material includes a magnetic material and the second conductive material includes an electrode material, and the size (diameter) of the cavity surrounded by the electrode layer on the concave side of the nearly hemispherical shell-shaped structure is in the range of about 10 nm to about 50 μm.

### TECHNICAL FIELD

The present invention relates to microstructures with structures such as hemispherical shells and semi-ellipsoidal shells, which are comprised of an adhesive material of a metal thin film and a conductive electrode thin film, and a method of detecting substances using the microstructures.

### BACKGROUND ART

Microstructures such as microparticles are widely used as materials for developing materials with novel physical properties, and as labeling materials for visualizing target proteins and DNAs in the life science field. Generally, spherical microparticles are widely used because they are easy to fabricate, but microparticles with complex shapes such as elliptical and polygonal microparticles have a wide range of applications because they have anisotropic optical properties.

In Japanese Patent Application Publication No. 2011-101941, “Hollow Microbody and Method for Producing The Same” (Patent Document 1), a method for producing hemispherical shell microparticles with a bowl-like shape is disclosed, and in WO2013/069732, “Magnetic Nanoparticles” (Patent Document 2), a method for producing hemispherical shell microparticles with magnetic materials and applying them to cell purification technology is disclosed.

Japanese Patent Application Publication No. 2011-101941 (Patent Document 1) discloses a method of producing hemispherical-shell microparticles by constructing a thin metal film by vacuum deposition or sputtering on polystyrene particles aligned on a flat substrate, and then removing the polystyrene particles by chemical treatment or heating. However, there is no indication of the specific application of the fabricated microparticles in the field of life science, especially the application of detecting biomolecules such as proteins and DNAs, which are important in medical diagnosis.

In WO2013/069732 (Patent Document 2), as one of the applications of the above-mentioned Japanese Patent Application Publication No. 2011-101941 (Patent Document 1), a method of purifying and recovering cells by size-selectively trapping cells in the inner hollow part of the microparticles, using magnetic materials such as nickel and iron to produce hemispherical shell microparticles of the same size as cells (approximately 10 μm in diameter), is disclosed. A method of producing hemispheric microparticles with superparamagnetic properties by using a multi-layered structure with an insulating layer between the magnetic thin films is also disclosed. However, no method has been shown for applications other than cell collection, especially for identifying the type and nature of cells by detecting biomolecules expressed on the surface of the collected cells.

Various methods have been developed for the detection of biomolecules, including fluorescence, luminescence, and electro-measurement. In particular, electrochemiluminescence (ECL) has attracted attention because it does not require excitation light, thus minimizing background light noise and is suitable for high-sensitivity measurement (low detection sensitivity of about 4 nM with affinity antibodies, 47 times higher than ELISA, 8 times higher than SPR (Liang et al., Assay Drug Dev. Technol., 5, 655, 2007 (Non-Patent Document 1))). ECL is a phenomenon in which an ECL probe, such as a ruthenium complex (Ru), and a co-reactant, such as tripropylamine (TPA), coexist in a few nm near the electrode, and emit light when a voltage is applied to the electrode resulting in high sensitivity. On the other hand, since ECL is a luminescence phenomenon that occurs only at a few nm near the electrode, it is incompatible with large micron-order objects such as cells, and there have been no research examples using cells as measurement targets.

Although various materials such as gold and silver are used as electrode materials, carbon, which has various conductive properties depending on the bonding mode between atoms, has been attracting attention as an electrode material. Graphene is a sheet-like material in which carbon atoms are arranged in a two-dimensional plane by sp² bonds, and it has attracted attention as an electrode material because of its extremely high electrical conductivity. However, because graphene is flat at the atomic level, constructing graphene films on curved microstructures with curvature requires technological innovation.

Nanocarbon thin films have been developed as a carbon thin film material with a graphene-like structure. Nanocarbon thin film is a thin film with a mixture of sp² and sp³ bonded regions prepared by unbalanced magnetron sputtering. It is a thin film material that is more stable in high humidity and high temperature than ordinary diamond-like carbon films, and has both high electrical conductivity (due to sp²) and diamond-like hardness (due to sp³) (JP 2006-90875 (Patent Document 3); Niwa et al., J. Am. Chem. Soc., 128, 7144, 2006 (Non-Patent Document 2); Jia et al., Anal. Chem., 79, 98, 2007 (Non-Patent Document 3). The surface of the nanocarbon thin film is flat at the atomic level and has a wide potential window for electrodes with low noise, which makes it superior to other carbon materials for electrochemical analysis and sensors. In fact, it has been demonstrated that nanocarbon thin film can be used as an electrode to measure total nucleobases and glial transmitters with high oxidation potential and low concentration with high reproducibility, which was difficult to detect with conventional electrodes (Kato et al., J. Am. Chem. Soc., 130, 3716, 2008 ((Non-patent document 4); Kato et al., Angew. Chem. Int. Ed., 47, 6681, 2008 (Non-patent document 5); Yamamura et al., Br. J. Pharmacol., 168 1088, 2013 (Non-patent document 6)). This nanocarbon deposition technology has been used on flat substrates such as silicon and glass. However, there have been no examples of deposition on three-dimensional materials such as hemispherical microstructures.

### CITATION LIST

### Patent Documents

### [Patent Document 1] Japanese Patent Application Publication No. 2011-101941

### [Patent Document 2] WO2013/069732

### [Patent Document 3] Japanese Patent Application Publication No. 2006-90875

### Non-Patent Documents

[Non-Patent Document 1] Liang et al., Assay Drug Dev. Technol. 5, 655, 2007
[Non-Patent Document 2] Niwa et al., J. Am. Chem. Soc., 128, 7144, 2006
[Non-Patent Document 3] Jia et al., Anal. Chem., 79, 98, 2007
[Non-Patent Document 4] Kato et al., J. Am. Chem. Soc., 130, 3716, 2008
[Non-Patent Document 5] Kato et al., Angew. Chem. Int. Ed., 47, 6681, 2008
[Non-Patent Document 6] Yamamura et al., Br. J. Pharmacol., 168 1088, 2013

### SUMMARY OF THE INVENTION

Therefore, it is desirable to provide a microstructure equipped with a mechanism to selectively detect marker molecules expressed by target cells or specific biomolecules, a method to fabricate the microstructure, and a specific solution to detect and identify molecules to be detected using the microstructure.

In view of the above circumstances, the present invention provides the following microstructures, a method for detecting molecules using the microstructures, and a method for producing the microstructures.

[1] A microstructure for use in the detection of molecules, comprising:
a nearly hemispherical shell-shaped structure made of a first conductive material, and
an electrode layer made of a second conductive material disposed on the concave side of the nearly hemispherical shell-shaped structure, wherein
the first conductive material comprises a magnetic material,
the second conductive material comprises an electrode material, and
the size (diameter) of the cavity surrounded by the electrode layer on the concave side of the nearly hemispherical shell-shaped structure is in the range of about 10 nm to about 50 μm.
   [2] The microstructure according to [1], wherein the cavity has a size (diameter) that is capable of receiving at least a single cell, and wherein the microstructure is used to detect biomolecules expressed on the surface of the cell.
   [3] The microstructure according to [2], wherein said biomolecules are molecules known to be expressed on the surface of a cancer cell and are used to identify the cancer cell.
   [4] The microstructure according to any one of [1] to [3], wherein the magnetic material comprises nickel, iron, or cobalt.
   [5] The microstructure according to any one of [1] to [4], wherein the electrode material comprises nanocarbon.
   [6] The microstructure according to any one of [1] to [5], wherein the microstructure has a magnetic property.
   [7] An array of the microstructures according to any one of [1] to [6], comprising a plurality of the microstructures oriented and arranged with the convex surface of the microstructures in contact with the electrode surface.
   [8] A method for detecting a molecule of interest using the microstructure according to any one of [1] to [6] or the array of the microstructures according to [7], comprising:
a) contacting a sample containing a test molecule suspected to contain the molecule of interest with an electrochemiluminescent probe, to specifically modify the molecule of interest with the electrochemiluminescent probe;
b) contacting the sample after step a) with the microstructure, to have the cavity surrounded by the electrode layer on the concave side of the microstructure receive the test molecule;
c) applying a voltage to the microstructure that has received the test molecule and observing the luminescence from the electrochemiluminescent probe; and
d) identifying the molecule of interest by detection of the luminescence.
   [9] The method according to [8], wherein specifically modifying a molecule of interest with the electrochemiluminescent probe comprises specifically binding to the molecule of interest an antibody that specifically binds to the molecule of interest, wherein the antibody is pre-labeled with the electrochemiluminescent probe.
   [10] The method according to [8] or [9], wherein the molecule of interest is a molecule known to be specifically expressed on the surface of a cancer cell, and wherein the sample is a sample solution containing test cells suspected of containing the cancer cell.
   [11] The method according to any one of [8] to [10], wherein the microstructure is magnetic, and the method further comprising a step of controlling the orientation of the microstructure by a magnetic field by applying an external magnetic field to the microstructure between step b) and step c).
   [12] The method according to [11], wherein the step of controlling the orientation of the microstructure by the magnetic field comprises arranging the microstructure in an orientation such that the convex surface of the microstructure is in contact with the electrode surface.
   [13] The method according to any one of [8] to [10], comprising a step of attaching the convex surface of the microstructure to a cantilever of an atomic electron microscope between step a) and step b).
   [14] A method for producing a nearly hemispherical shell-shaped microstructure, comprising steps of:
a) preparing nearly hemispherical mold microparticles of a desired size disposed in a monolayer on a substrate, wherein the mold microparticles are made of a material that can be removed by a predetermined removal process;
b) coating the mold microparticles disposed on the substrate in the monolayer with a second conductive material;
c) further coating the mold particles coated with the second conductive material with the first conductive material; and
d) removing the mold microparticles by the predetermined removal process to obtain microstructures having a nearly hemispherical shell-shaped structure made of the first conductive material and an electrode layer made of the second conductive material disposed on the concave side of the nearly hemispherical shell-shaped structure,
wherein the first conductive material comprises a magnetic material,
the second conductive material comprises an electrode material, and
the size (diameter) of the mold microparticle is in the range of about 10 nm to about 50 μm.
   [15] The method according to [14], further comprising the step of further coating the mold particles coated with the second conductive material with the third conductive material between step b) and step c), and in step c), further coating the mold particles coated with the third conductive material with the first conductive material.
   [16] The method according to [14] or [15], wherein the step of coating the mold microparticles with the first, second or third conductive material comprises coating the mold microparticles using a thin film deposition device selected from the group consisting of a sputtering device, a resistance heating vacuum deposition device, and a chemical vapor deposition device.
   [17] The method according to any one of [14] to [16], wherein the magnetic material comprises nickel, iron, or cobalt.
   [18] The method according to any one of [14] to [17], wherein the electrode material comprises nanocarbons, and the thin film formed in the step of coating with the second conductive material comprises a nanocarbon thin film with a mixture of sp²-bonded regions and sp³-bonded regions.
   [19] The method according to any one of [14] to [18], wherein the material forming the mold particles comprises a material selected from the group consisting of polystyrene, polypropylene, cellulose, and glass.
   [20] The method according to any one of [14] to [19], wherein the predetermined removal process comprises removing the mold particles by a process selected from the group consisting of heating the mold particles to a high temperature, treating the mold particles with an organic solvent, and treating the mold particles with active oxygen.
   [21] The method according to [20], wherein the predetermined removal process comprises heating at a high temperature in an atmosphere with an oxygen concentration of about 15% or less.
   [22] The method according to any one of [14] to [21], wherein the cavity surrounded by the electrode layer of the concave surface of the nearly hemispherical shell-shaped structure has a size (diameter) that is capable of receiving at least a single cell.
   [23] The method according to any one of [14] to [22], wherein the thickness of each thin film layer formed in the step of coating the mold microparticles with the first, second, or third conductive material is in the range of about 0.1 nm to about 1 mm.

In other words, the present invention provides a method of producing a hemispherical shell (or shell-shaped) microstructure, characterized by arrangement of electrodes on the concave side, made of a thin metal film of the thickness and diameter desired to be produced, and a method of detecting target biomolecules using the same. The present invention also provides a method for producing and controlling a microstructure in which the outer surface of the electrode microstructure described above includes a magnetic material and the orientation of the microstructure can be controlled by applying an external magnetic field, and a method for producing a magnetic microstructure described above by performing a mold particle removal reaction in an environment with a low oxygen concentration (e.g., less than about 15%), thereby providing the microstructure with high magnetic field responsiveness, a method of producing the microstructure in which the electrode material of the electrode microstructure above is a thin film of nanocarbon in which sp² and sp³ binding regions are mixed and which can be formed on a curved surface, a method of capturing a biomolecule or cell inside the microstructure by orientating and arranging the electrode microstructure on a flat substrate, and a method of capturing a biomolecule or cell inside the microstructure using the electrode microstructure dispersed in a solution, and controlling the orientation of the microstructure by applying an external magnetic field.

Furthermore, a method of detecting a target molecule by an electrochemical luminescence reaction by applying a voltage to a biomolecule or cell trapped in the electrode microstructure is provided.

The advantage of the microstructure of the present invention is that it can employ electrochemiluminescence for the detection of biomolecules, which eliminates the need for excitation light, suppresses background light noise, and enables highly sensitive measurements.

When the microstructure of the present invention is used to detect biomolecules by receiving a cell or biomolecules in the cavity on the concave side of the microstructure, the electrode can be placed in contact with a larger area of the cell surface compared to a conventional electrode placed on a flat substrate. The electrode can be placed in closer proximity to the electrochemiluminescent probes bound to the biomolecules expressed on the cell surface, which significantly increases the sensitivity of signal detection from the probes. In particular, when the microstructure of the present invention, which has a concave surface with a curved surface such as a hemispherical or semi-ellipsoidal shape, is used for the detection of biomolecules on the cell surface, the effect can be enhanced because the microstructure is shaped to better follow the curved surface of the cell surface.

The detection of biomolecules on the cell surface using the microstructures of the present invention makes it easier to detect signals from biomolecules on the cell surface, which were previously difficult to detect due to obstruction (or shielding) by the three-dimensional structure of the cell itself.

Applying an external magnetic field to the magnetic microstructure of the present invention makes it easier to control the orientation of the microstructure (e.g., orientation arrangement), and as a result, cells can be captured on the concave side of the microstructure in the solution phase first, which is expected to significantly improve the cell capture rate on the microstructure. In addition, since it is easier to attach the microstructure to the electrode surface after capturing cells in the solution phase, it is easier to apply a voltage to the microstructure, which in turn makes it easier to detect the cells or biomolecules received on the concave surface of the microstructure using a probe label.

### BRIEF DESCRIPTION OF THE DRAWINGS

FIG. 1 shows an example of a two-layered electrode microstructure.

FIG. 2 shows an example of how to fabricate an electrode microstructure.

FIG. 3 shows the relationship between the oxygen concentration and the magnetic field response (the percentage of microstructures recovered by applying a magnetic field) when removing the mold particles.

FIG. 4 shows an example of a method for detecting target molecules on the cell surface by electrochemiluminescence (ECL) using electrode microstructures. FIG. 4-1 shows an example of a method to measure ECL with electrode microstructures arranged in an array. FIG. 4-2 shows an example of a method to measure ECL by attaching an electrode microstructure to the tip of a micro-needle. FIG. 4-3 is a schematic diagram showing examples of the following methods: (a) dispersing the electrode microstructure in the solvent in a conductive tube and capturing target cells and molecules in the solvent, (b) performing ECL measurement after applying a magnetic field from outside the conductive tube, (c) performing ECL measurement by dropping the solution in the tube onto the electrode substrate, and a micrograph (d) of the experimental results of the method of ECL measurement by dropping the solution in the tube onto the electrode substrate.

FIG. 5 shows a schematic diagram of the method of labeling the target molecules on the cell surface with ECL probe-labeled antibodies and measuring ECL.

FIG. 6 shows (a) a micrograph and graph showing the results of an experiment in which hemispherical shell electrode microstructures with a diameter of 15 μm were arrayed on a conductive adhesive material (silver) with a two-layered structure consisting of a nanocarbon thin film on the inner surface and nickel on the outer surface, and EpCAM molecules on the surface of MCF-7 cells, which are EpCAM high-expressing cancer cells, were labeled with ruthenium-labeled antibodies and then captured in the microstructures and voltage was applied to measure ECL; (b) a micrograph showing the results of an experiment in which EpCAM molecules on the surface of MDA-MB-231 cells, which are EpCAM low-expressing cancer cells, were similarly labeled and then trapped within the microstructure, voltage was applied, and ECL measurements were performed; (c) a micrograph showing the results of an experiment in which, as a comparison, a ruthenium-labeled antibody against GAPDH, which is not expressed on the cell surface, was reacted with MDA-MB-231 cells and ECL measurements were performed in the same way; and (d) a graph showing the results of comparing the number of bright spots in the ECL measurements in (b) and (c).

FIG. 7 shows fluorescence micrographs and graphs of ECL measurements of hemispherical shell electrode microstructures with a diameter of 15 μm, consisting of two layers of nanocarbon thin film on the inner surface and nickel on the outer surface, placed in an array on a conductive adhesive (silver), with 2 nM, 2 μM, and 2 mM ruthenium complexes added in the solvent in the presence of 1 mM concentration of tripropylamine.

### EMBODIMENTS FOR IMPLEMENTING THE INVENTION

### 1. Microstructures for Use in the Detection of Molecules

In one aspect, the present invention provides microstructures for use in the detection of molecules (sometimes referred to herein as “microstructures of the present invention” or “electrode microstructures,” “hemispherical shell-shaped microstructures,” etc.). In one typical embodiment, the first conductive material includes a magnetic material, and the second conductive material includes an electrode material.

Examples of the first conductive material include, but are not limited to, magnetic materials such as metals such as nickel, iron, and cobalt, oxides such as iron oxide and chromium oxide, or alloys such as ferrite and neodymium.

With respect to the present invention, when referring to “magnetic material,” the term “magnetic material” is used in its ordinary meaning as used in the art. For the purpose of the present invention, at least the “magnetic material” used in the present invention should be magnetic to the extent that when an external magnetic field is applied, the orientation of the microstructure can be controlled by the magnetic field.

As used herein, “molecule” includes both specific target molecules dispersed in a solvent and biomolecules expressed on a cell surface. Non-limiting examples of biomolecules expressed on the cell surface include molecules expressed on the surface of cancer cells such as EpCAM, epidermal growth factor receptor (EGFR), programmed cell death ligand-1 (PD-L1), and cadherins.

The “cells” are typically cells obtained from mammals, including humans (e.g., humans, cattle, pigs, goats, sheep, monkeys, dogs, cats, mice, rats, etc.), but may also include, but are not limited to, cells from birds, reptiles, amphibians, insects, microorganisms, plants, etc.

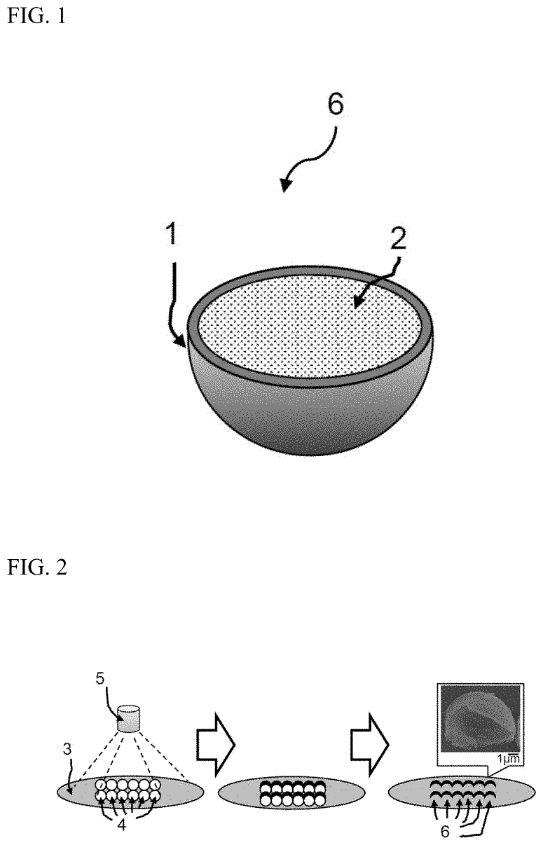

FIG. 1 shows an example of the electrode microstructure of the present invention. In this example, a two-layered hemispherical shell-shaped microstructure 6 consisting of a metal thin film 1 as the first conductive material and an electrode thin film 2 as the second conductive material is shown, but the number of layers is not limited to two, and multiple thin film layers of different elements or elemental alloys may be sandwiched as intermediate layers. The type of thin-film element must be a conductive element such as a metal in the case of electrochemiluminescence measurement, but is not limited to this category in other cases. The thickness of the thin film can be freely selected within the range where the structure of the microstructure can be maintained, and the thickness per layer is in the range of about 0.1 nm to 1 mm, most preferably 1 nm to 1 μm. The shape of the microsphere can be freely fabricated according to the shape of the mold used to fabricate the microsphere, and can be hemispherical, cylindrical, pyramidal, semi-ellipsoidal, prismatic, or pyramidal, but is not limited to this range. It will be understood from the description herein that, for example, the mold microparticles themselves for creating the hemispherical shell-shaped microstructure 6 do not necessarily have to be hemispherical, but may be spherical. As used herein, the terms “nearly hemispherical,” “nearly hemispherical-shelled,” or “nearly spherical” shall, unless otherwise noted, include all the shapes or shell shapes illustrated herein, as well as those with distortions of shape that may be acceptable in actual manufacturing situations. The size (diameter) of the concave side cavity of the hemispherical shell-shaped structure of this micro body can also be freely fabricated according to the shape of the mold, and ranges from about 1 nm to about 1 cm, preferably from about 1 nm to about 500 μm, more preferably from about 5 nm to about 100 μm, and most preferably from about 10 nm to about 50 μm. Typically, the size of this cavity can be of a size (diameter) that is capable of receiving at least a single cell.

### 2. Method for Manufacturing Microstructures for Use in Molecular Detection.

The present invention also provides, in another aspect, a method for manufacturing the microstructures of the present invention. This manufacturing method comprises.
a) preparing nearly spherical mold particles of a desired size disposed in a single layer on a substrate;
b) coating the mold particles disposed on the substrate in the single layer with a second conductive material;
c) further coating the mold particles coated with the second conductive material with a first conductive material; and
d) removing the mold microparticles by the predetermined removal process to obtain a microstructure having a nearly hemispherical shell-shaped structure made of the first conductive material and an electrode layer made of the second conductive material disposed on the concave side of the nearly hemispherical shell-shaped structure.

Typically, the mold microparticles comprise a material that can be removed by a predetermined removal process.

The typical requirements for the first and second conductive materials and the typical size of the mold microparticles or the concave side cavities of the hemispherical shell-shaped structure have already been described in Section 1 above with respect to the microstructures of the present invention.

FIG. 2 shows an example of a specific production method of the electrode microstructure 6 of the present invention. First, a single layer of microparticles 4, which serve as a mold, is placed on a planar substrate 3. The material of the planar substrate can be glass, silicon, plastic, etc. However, as long as the substrate has a surface planarity smaller than the size of the mold microparticles, it is not limited to these materials and any substrate can be used. As for the mold particles, polystyrene particles, polypropylene particles, cellulose particles, glass particles, etc. can be used, but they are not limited to these as long as the particles have the equivalent size and shape of the electrode microstructure to be produced. For example, when placing a single layer of polystyrene particles with a diameter of 10 μm on the surface of a glass slide substrate, place about 100 μL of commercially available polystyrene particle dispersion solution in a tube and centrifuge it at 1,500×G for about 5 minutes to precipitate the particles, then discard the supernatant and add a highly volatile solvent such as water or ethanol to the tube to redisperse the particles. After the particles are redispersed, the particle dispersion solution can be dropped onto a clean glass slide substrate and dried.

In order to form the inner electrode thin film 2 (made of the second conductive material) of the electrode microstructure 6 shown in FIG. 1, the planar substrate on which the mold microparticles are placed is placed in a sample chamber of the thin film formation system 5, and the thin film that serves as the electrode material is prepared at an arbitrary film thickness according to the operating procedure of the thin film formation system. As a result, a thin film of electrode material is formed on the mold particles placed in a single layer on the planar substrate. Electrode materials (or electrode materials) include but are not limited to carbon, gold, silver, copper, aluminum, nickel, transparent conductive materials such as indium tin oxide (ITO), and conductive polymers such as PEDOT, as long as the material has an electrical resistivity of about 1 ohm·cm or less. For example, when a thin film is formed using the above procedure on polystyrene particles placed on a glass substrate, the entire particle is not coated with the thin film, but only the hemispherical portion facing the direction of the elemental material of the thin film forming device (not facing the substrate) is coated with the thin film. The thin film forming device can be a sputtering device, a resistance heating vacuum deposition device, or a chemical vapor deposition device, but it is not limited to any of these as long as the device is capable of forming a thin film of any range within a thickness of about 0.1 nm to about 1 mm, which is less than or equal to the size of the mold particles. In the case of using nanocarbon thin film as electrode thin film material, an unbalanced magnetron sputtering system that can form carbon thin film with a mixture of sp² and sp³ bonded regions is used.

Nanocarbon thin film is a carbon thin film with a mixture of sp² and sp³ bonded regions. The sp² bonded region can bind molecules with a cyclic structure such as pyrene, nanographene, and DNA through π-π bond interactions, and the existence of the sp³ bonded region makes it possible to form a continuous film on curved surfaces such as hemispherical microstructures. The ratio of sp² to sp³ bonded regions can be freely adjusted depending on the sputtering conditions. For example, when forming a nanocarbon thin film on a micro body with a curvature such as polystyrene particles (e.g., with a particle size of about 10 nm to 50 μm), if the sputtering conditions are set so that the sp²:sp³ ratio is about 8:2, a nanocarbon thin film with relatively soft composition can be formed on the micro body with curvature while maintaining the surface planarity.

Next, in order to form the metal thin film 1 (made of the first conductive material) on the outer surface of the electrode microstructure 6 in FIG. 1, the sample is placed in the sample chamber of the thin film deposition system 5, and the thin film is formed in exactly the same way as when forming the electrode thin film. As an example, in the case of polystyrene particles with a single layer of nanocarbon thin film placed on a glass substrate, 100 nm of nickel thin film is deposited on top of the nanocarbon thin film using a vacuum deposition system. This results in a two-layer thin film being formed on the hemispherical portion of the mold particle. If the number of thin film layers is to be increased to three or more, repeat this procedure to increase the number of layers.

After forming a multi-layered thin film on the mold particles using the above procedure, the mold particles are removed to obtain the electrode microstructure 6 as shown in FIG. 1. The removal method of the mold particles may include high temperature heating, organic solvent treatment, and active oxygen treatment. For example, if a nanocarbon thin film and a nickel thin film are formed in a single layer on a glass substrate, the polystyrene particles placed on the glass substrate are then placed in an electric furnace chamber and heat-treated at 500° C. for one hour to remove the mold polystyrene particles. A two-layered hemispherical microstructure consisting of the nanocarbon thin film and the nickel thin film is then produced on the glass substrate, with the aperture facing the glass substrate side. Here, heating treatment using an electric furnace is given as an example, but it is not limited to this technique as long as the mold particles are removed, and the thin film layer is not removed. For example, if glass microparticles are used as a mold, the glass microparticles are placed on a polyethylene or Teflon substrate, an electrode and a metal thin film are formed, and then hydrogen fluoride treatment is performed to remove the glass microparticles.

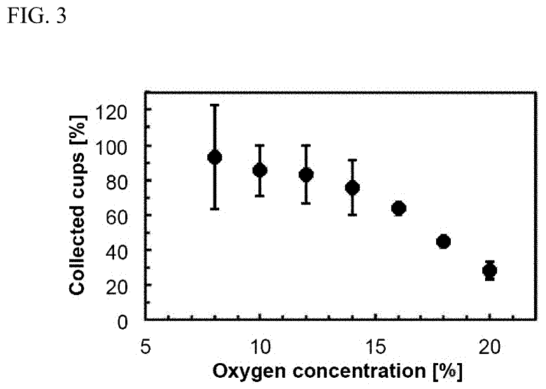

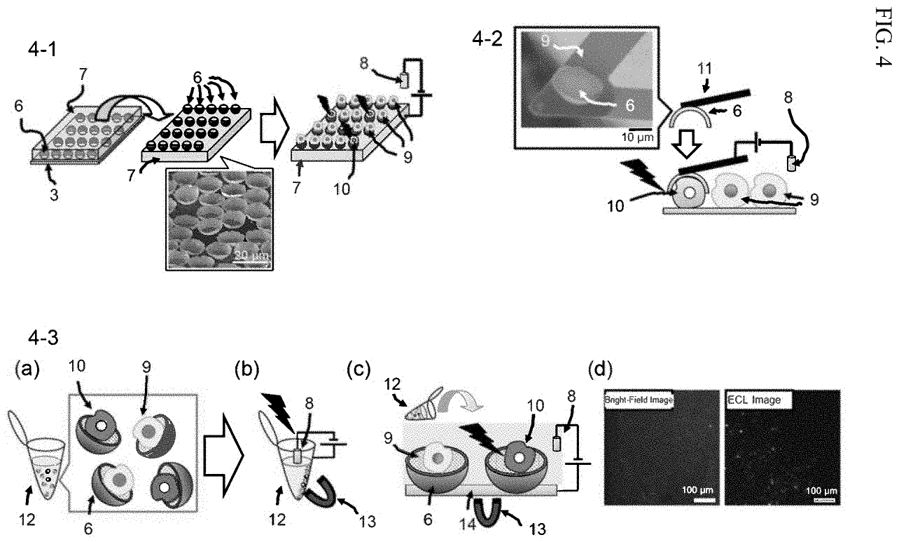

If it is desired to produce a magnetic microparticle, it is recommended that the removal process of the mold particles be performed in an atmosphere with a low oxygen concentration (typically, no more than about 15%) to maintain the magnetic field response, as shown in FIG. 3. For example, in the case of polystyrene particles with a single layer of a nanocarbon thin film and a nickel thin film on a glass substrate, if the polystyrene particles are to be removed by heating while maintaining the magnetic moment of the nickel thin film, the small electric furnace is placed in a glove box or other atmosphere changeable box, and nitrogen gas is introduced into the box. By introducing nitrogen gas into the box to reduce the oxygen concentration in the box to about 15% or lower (e.g., about 14%, about 13%, about 12%, about 11%, about 10%, about 9%, about 8%, about 7%, about 6%, about 5%, or lower), and then starting the heating process in the electric furnace, the magnetic moment is maintained. In this way, magnetic microstructures with maintained magnetic moment can be produced. Although the optimal oxygen concentration that can be used may vary depending on the environment, equipment, materials, etc., those skilled in the art will be able to determine the optimal oxygen concentration for maintaining the magnetic moment in the microstructure based on the teachings herein and common general knowledge in the art. Such oxygen concentrations include, for example, a concentration selected from the group consisting of about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, and about 20%, or a concentration range between any two concentrations selected therefrom.

### 3. A Method for Detecting Molecules Using the Microstructure of the Present Invention.

In yet another aspect, the present invention provides a method of detecting a molecule of interest using at least one microstructure of the present invention or an array thereof, or at least one microstructure of the present invention or an array thereof produced by a method of producing a microstructure of the present invention. This method typically includes
a) contacting a sample containing a test molecule suspected of containing the molecule of interest with an electrochemiluminescent probe to specifically modify the molecule of interest with the electrochemiluminescent probe;
b) contacting the sample after the step a) with the microstructure, to have the test molecule received in the cavity surrounded by the electrode layer on the concave side of the microstructure;
c) applying a voltage to the microstructure that has received the test molecule and observing the luminescence from the electrochemiluminescent probe; and
d) identifying the molecule of interest by detection of the luminescence.

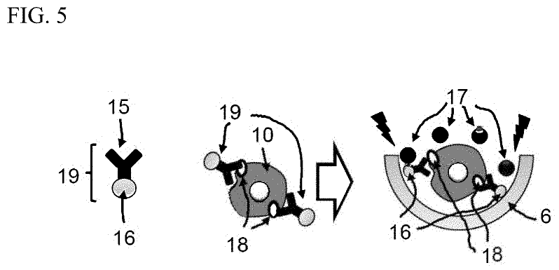

As used herein, “array” is used in the sense normally used in the art, and when referring to an “array of microstructures” with respect to the present invention, it means a population of microstructures in which two or more microstructures are arranged in one or two dimensions (see, for example, FIG. 4-1).

There are three possible uses of the produced electrode microstructures for the detection of biomolecules by electrochemiluminescence (ECL) method.

As shown in FIG. 4-1, the first use is to arrange electrode microstructures 6 in an array on a conductive flat substrate with the openings exposed, and to detect biomolecules by applying a voltage between the microstructures and the solvent after bringing the biomolecules close to the electrode portions in the microstructure openings. Here, a method of detecting only cancer cells from a mixture of normal cells 9 and cancer cells 10 by ECL using a microstructure in the shape of a hemispherical cup with a two-layered structure of nanocarbon thin film on the inner surface and nickel on the outer surface (hereinafter referred to as “electrode cup”) as a detection target for receptor molecules on the surface of cancer cells is explained as a specific example. The following is an explanation of the method.

First, for the electrode cup prepared with the opening facing the glass substrate side by heat treatment, a conductive adhesive material 7 can be applied from above and peeled off as shown in FIG. 4-1, so that the electrode cup 6 can be peeled off from the glass substrate 3 and transferred onto the adhesive material 7. The conductive adhesive material can be a tape made of carbon, gold, silver, copper, aluminum, etc., or a light-curing transparent conductive polymer such as PEDOT. When a cell suspension containing cancer cells is dropped on top of this, the individual cells are trapped in the concave depression of the electrode cup. Since the larger the contact area between the cells and the electrode cup, the greater the amount of signal during ECL measurement, the diameter of the electrode cup should be approximately the same as that of the cells (about 10 μm in diameter), typically 10-15 μm in diameter in this example. Also, the suspension of cells containing cancer cells is pre-modified with ECL probe molecules to detect cancer cells by ECL measurement, the specific procedure of which will be described later. After each cell is trapped on the concave surface of the electrode cup by the above procedure, when a voltage is applied between the conductive adhesive material that conducts with the electrode cup and the cell suspension, ECL emission is observed from the electrode cup trapping the cancer cell labeled with ECL probe molecule, and the presence of the cancer cell is thus identified. The applied voltage is typically swept in the range of 0-2V. A specific example of a cell suspension containing cancer cells would be the blood of a cancer patient, which could be used for diagnostic applications such as detecting circulating cancer cells in the blood.

The second type of use is a method of approaching the electrode microstructure 6 on the cells on the substrate by attaching the electrode microstructure 6 to the tip of a fine needle, as shown in FIG. 4-2. Here, a method of performing ECL measurement using a cantilever of an atomic force microscope (AFM) with an electrode cup attached to the tip is described as a specific example.

A conductive cantilever with a metal-coated surface, a conductive adhesive, and an electrode cup detached from the substrate are each placed under a stereomicroscope equipped with a micromanipulator. For the conductive cantilever, a commercial AFM cantilever coated with gold or other metals may be used. For the conductive adhesive, commercially available silver paste adhesive may be used. When peeling off the electrode cup from the substrate, the cup can be dispersed in the solvent by dropping about 100 μL of a solvent such as water or ethanol onto the electrode cup on the substrate, which has been prepared by heat treatment with the opening facing the glass substrate, and applying ultrasonic waves from the bottom side of the substrate. To apply ultrasonic waves, place the electrode cup in a Petri dish with the glass substrate, place the bottom of the Petri dish against the water surface of the ultrasonic cleaner, and run the ultrasonic cleaner. While observing with a stereomicroscope, touch the tip of the glass needle of the micromanipulator to the conductive adhesive, and the adhesive will adhere to the tip of the glass needle. When the micromanipulator's glass needle is touched to the tip of the AFM cantilever, a very small amount of conductive adhesive sticks to the tip of the cantilever. Next, a new glass needle is prepared, and when it touches the electrode cup, a single piece of the electrode cup adheres to the glass needle tip of the micromanipulator due to electrostatic interaction. When it is touched to the part of the AFM cantilever tip to which the adhesive is attached, the single piece of electrode cup adheres to the cantilever tip through the adhesive. The conductive AFM cantilever to which the electrode cup is attached is placed in the AFM system. The cell to be measured is a cancer cell pre-labeled with an ECL probe. When the conductive AFM cantilever with the electrode cup attached is attached to the cell surface in the same way as in the normal AFM approach operation, and a voltage is applied between the AFM cantilever and the solvent, ECL luminescence is observed only when the AFM cantilever with the electrode cup approaches the ECL probe-labeled cancer cell.

The third type of use is to disperse the electrode microstructure 6 in a solvent and perform ECL measurement after capturing the target cells and target molecules in the solvent, as shown in FIG. 4-3. Here, the method of detecting cancer cells by mixing a magnetic electrode cup with a cell suspension is explained as a specific example.

A drop of about 100 μL of a solvent such as cell culture medium is dropped onto an electrode cup on a substrate prepared by heat treatment with the opening facing the glass substrate side, and the cup is dispersed in the dropped solvent by applying ultrasonic waves from the bottom side of the substrate. The cup dispersion and the cell suspension containing cancer cells are transferred to a vessel with a conductive wall, mixed, and allowed to rotate or shake for 30 minutes to 1 hour to trap the cells in the concave depression of the electrode cup, as shown in FIG. 4-3(a). Cancer cells can be pre-labeled with ECL probe molecules, and commercially available tubes with metal-coated inner surfaces can be used as the vessel with conductive walls. When a magnetic field is applied from the outside of the vessel after the cells are sufficiently trapped in the concave depression of the electrode cup, as shown in FIG. 4-3(b), the electrode cup with the trapped cells adheres to the inner wall of the vessel because the electrode cup is magnetic. When a voltage is applied between the conductive inner wall of the vessel and the solvent, ECL luminescence is observed from the ECL probe-labeled cancer cells.

Alternatively, as shown in FIG. 4-3(c), after the cells are fully trapped in the electrode cup concave depressions, the solution in the tube is dropped onto the electrode substrate 14 and a magnetic field is applied from the back side of the electrode substrate, the electrode cup trapping the cells accumulates on the electrode substrate because the electrode cup is magnetic. When a voltage is applied between the electrode substrate and the solvent, ECL luminescence is observed from the ECL probe-labeled cancer cells. The tube does not need to be conductive to use this method. The electrode substrate can be any conductive substrate, including metals such as gold, silver, copper, and aluminum, and transparent electrode materials such as ITO.

FIG. 4-3(d) shows an example of an experiment that implements the example shown in FIG. 4-3(c). Here, cancer cells were pre-labeled with antibodies against epithelial cell adhesion molecules with ECL substrates as described below, mixed with cup-shaped electrode microstructures with a diameter of 15 μm, and then the solution containing the cells trapped in the cups was dropped onto a silver electrode substrate, and the cells were trapped on the electrode by applying a neodymium magnet from the back side of the electrode. Each starting point in the ECL image is the ECL luminescence from an individual cup that has captured cancer cells.

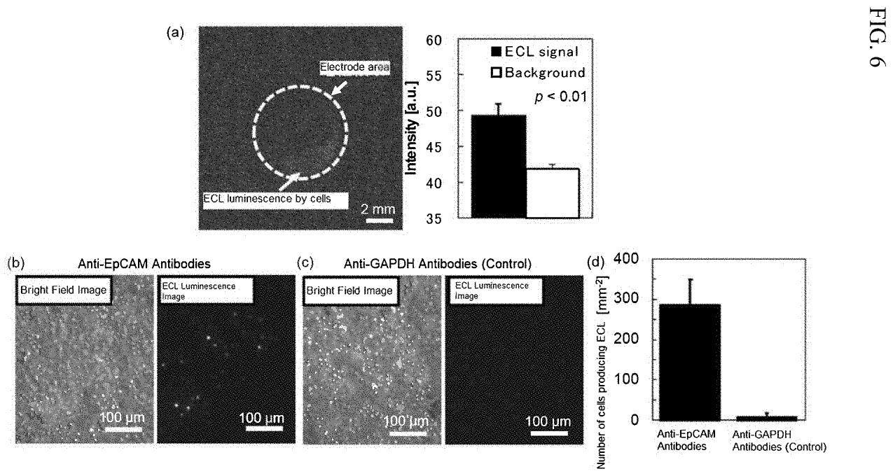

As shown in FIG. 5, there is a method of detecting ECL probe-labeled target molecules on the cell surface by using antibodies 15 that selectively bind to the target molecules. Here, epithelial cell adhesion molecule (EpCAM) expressed on the surface of cancer cells is used as the target molecule for detection, ruthenium complex (Ru) as the ECL probe, and tripropylamine (TPA) as the ECL probe. tripropylamine (TPA) as co-reactants in ECL measurement, and an electrode cup is used to detect cancer cells by ECL measurement as a specific example. When a voltage is applied between the electrode and the solvent with the ECL probe Ru and the co-reactant TPA coexisting in a region of several nm near the electrode, ECL emission is observed.

By mixing Ru-labeled anti-EpCAM antibodies with a cell suspension containing cancer cells and rotating or shaking the reaction for 30 minutes to 1 hour, Ru-labeled anti-EpCAM antibodies bind to EpCAM on the surface of cancer cells, resulting in Ru labeling of the cancer cell surface. For Ru labeling of the antibodies, Ru containing N-hydroxysuccinimide (NHS), which reacts with an amino group, is commercially available, and by mixing this reagent with the antibodies, the amino group of the antibody binds to Ru, resulting in a Ru-labeled antibody. In the cell suspension containing Ru-labeled cancer cells, TPA is added to the solvent, and the cells are trapped in the electrode cup by any of the three methods described above, and then a voltage is applied between the electrode cup and the solvent. In the electrode cup containing a cancer cell labeled with Ru-labeled anti-EpCAM antibody, Ru and TPA (present in the solvent) coexist near the nanocarbon electrode on the concave surface of the cup, and ECL emission is observed when voltage is applied. On the other hand, in an electrode cup trapping normal cells such as leukocytes that do not express EpCAM, ECL luminescence does not occur because of absence of Ru, although TPA is present. As a result, ECL luminescence is observed only from the electrode cup in which a cancer cell is captured, leading to the detection of the cancer cell. FIG. 6 shows an example of the above ECL measurement of cancer cells in practice, and the presence of cancer cells is confirmed as ECL luminescence. FIG. 6(b) shows the results of ECL measurement for MDA-MB-231 cells, which are known to be difficult to detect by fluorescence. The detection of cancer cells, which are known to be difficult to detect by fluorescence, was also successfully achieved by the present method, demonstrating the high detection sensitivity of the present method. In this way, the present invention enables highly sensitive detection of cell surface target molecules by ECL measurement, which has been difficult to achieve before.

If there is a concern that the addition of TPA to the solvent may affect cell activity, it is possible to chemically synthesize a reagent in which TPA is covalently linked to Ru along with NHS. In this case, the reaction of the reagent with anti-EpCAM antibody yields Ru-TPA-labeled anti-EpCAM antibody, which is labeled with both Ru and TPA. When this antibody is reacted with a cancer cell, the surface of the cancer cell is labeled with both Ru and TPA. After the cancer is trapped in the electrode cup by any of the three methods described above, ECL emission is observed when a voltage is applied between the electrode cup and the solvent. In this case, there is no need to add TPA to the solvent, and any solvent can be used.

Although the above example specifically illustrates EpCAM as a molecule expressed on the surface of cancer cells, it will be obvious to those skilled in the art that other molecules known to be expressed on the surface of cancer cells can be detected in the same manner as described above. Examples of such other molecules include, but are not limited to, epidermal growth factor receptor (EGFR), programmed cell death ligand-1 (PD-L1), cadherin, etc.

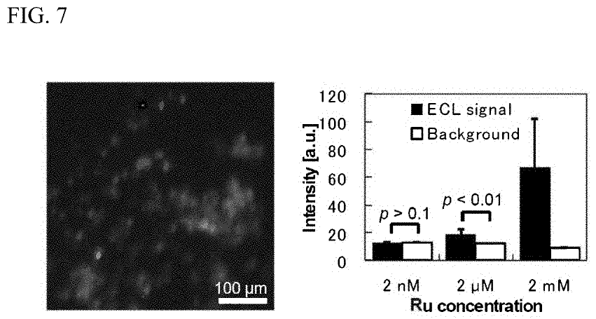

In the case of detecting specific target molecules dispersed in a solvent instead of cell surface molecules such as EpCAM, ECL probe molecules that bind to the target molecules are mixed in the solvent, as in the case of cell surface measurement, to bind the target molecules to the ECL probe molecules, and then unreacted ECL probe molecules are removed by column purification, etc. The ECL measurement can then be performed by dropping the solution in which the conjugate of the target molecule and the ECL probe molecule is dispersed onto the electrode microstructure 6. For example, in the case of detecting avidin, which is a protein, biotin that has strong binding ability to avidin can be labeled with ECL probe such as Ru, and then Ru-labeled biotin can be mixed with avidin and bound to it for ECL measurement. An example of ECL measurement is shown in FIG. 7, where Ru itself is used as a target molecule for detection, and a solvent containing 1 mM concentration of TPA is dropped onto the electrode cup, and 2 nM to 2 mM concentration of Ru is added to the solvent, and a voltage is applied between the electrode cup and the solvent. As shown in FIG. 7, we have confirmed that 2 μM concentration of Ru is detected as ECL emission from each electrode cup.

In the above example, the case of using a hemispherical shell electrode cup, EpCAM on the surface of cancer cells as the detection target, Ru as the ECL probe molecule, and TPA as the co-reactant are shown as specific examples. However, the skilled person would easily assume that the measurement method is the same even if the shape of the electrode microstructure 6, the molecule to be detected, the type of ECL probe molecules and co-reactants used, etc. are different. As for the molecules to be detected, biomolecules such as proteins, peptides, nucleic acids, and secretory vesicles of cells are assumed to be the targets, but any molecules that can be labeled with ECL probes can be detected by this measurement method.

The technical scope of the present invention is not limited to these specific examples, but various variations are possible within the technical scope of the invention and its equivalents described in the appended claims, and these variations are also included in the technical scope of the present invention.

### INDUSTRIAL APPLICABILITY

To be used in the field of liquid biopsy to detect marker molecules and marker cells in blood, a diagnostic device or a diagnostic chip can be developed to detect marker molecules that are expressed only by cancer cells and not by normal blood cells using the present invention technology, which can be applied to the diagnosis of cancer cells circulating in blood. By preparing a chip with the microstructures of the present invention placed on a substrate and dropping blood onto it, it can be used as a chip for detecting cancer cells circulating in the blood.

The microstructures of the present invention can also be applied to the detection of certain substances and microorganisms in the environment. Similar to the above blood-circulating cancer cell detection chip, the electrode microstructures of the present invention can be used as a simple environmental inspection chip by placing them on a substrate and detecting viruses and specific chemical substances.

The microstructures can be dispersed in a solution, and after dispersion, they can be integrated and oriented in a solution by applying an external magnetic field, so they can be used for the manufacture of new conductive and magnetic materials.

### DESCRIPTION OF REFERENCE CODES

1: Metal thin film, 2: Electrode thin film, 3: Flat substrate, 4: Mold particles, 5: Thin film formation device, 6: Electrode microstructure, 7: Conductive adhesive material, 8: Reference electrode, 9: Normal cell, 10: Cancer cell, 11: Atomic force microscope cantilever, 12: Tube, 13 Magnet, 14 Electrode substrate, 15: Antibody, 16: ECL probe such as ruthenium, 17: Co-reactant such as tripropylamine, 18: Cell surface target molecule, 19: ECL probe modified antibody.

1. A microstructure for use in the detection of molecules, comprising:
a nearly hemispherical shell-shaped structure made of a first conductive material, and
an electrode layer made of a second conductive material disposed on the concave side of the nearly hemispherical shell-shaped structure, wherein
the first conductive material comprises a magnetic material,
the second conductive material comprises an electrode material, and
the size (diameter) of the cavity surrounded by the electrode layer on the concave side of the nearly hemispherical shell-shaped structure is in the range of about 10 nm to about 50 μm.

2. The microstructure according to claim 1, wherein the cavity has a size (diameter) that is capable of receiving at least a single cell, and wherein the microstructure is used to detect biomolecules expressed on the surface of the cell.

3. The microstructure according to claim 2, wherein said biomolecules are molecules known to be expressed on the surface of a cancer cell and are used to identify the cancer cell.

4. The microstructure according to claim 1, wherein the magnetic material comprises nickel, iron, or cobalt.

5. The microstructure according to claim 1, wherein the electrode material comprises nanocarbon.

6. The microstructure according to claim 1, wherein the microstructure has a magnetic property.

7. An array of the microstructures according to claim 1, comprising a plurality of the microstructures oriented and arranged with the convex surface of the microstructures in contact with the electrode surface.

8. A method for detecting a molecule of interest using a microstructure or an array thereof, the method comprising:
a) specifically modifying the molecule of interest with an electrochemiluminescent probe by contacting a sample containing the molecule of interest with the electrochemiluminescent probe,
b) contacting the molecule of interest modified with the electrochemiluminescent probe obtained in step a) with a microstructure, to receive the test molecule in a cavity of the microstructure;
c) applying a voltage to the microstructure that has received the molecule of interest; and
d) detecting the molecule of interest by observing of the luminescence from the electrochemiluminescent probe,
wherein the microstructure comprises:
a nearly hemispherical shell-shaped structure made of a first conductive material, and
an electrode layer made of a second conductive material disposed on the concave side of the nearly hemispherical shell-shaped structure, and wherein
the first conductive material comprises a magnetic material,
the second conductive material comprises an electrode material, and
the cavity is formed by being surrounded by the electrode layer on the concave side of the microstructure, and the size (diameter) of the cavity is in the range of 10 nm to 50 μm.

9. The method according to claim 8, wherein specifically modifying the molecule of interest with the electrochemiluminescent probe comprises: (a) binding the molecule of interest to the electrochemiluminescent probe, or (b) specifically binding to the molecule of interest an antibody that specifically binds to the molecule of interest, wherein the antibody is pre-labeled with the electrochemiluminescent probe.

10. The method according to claim 8, wherein the molecule of interest is a molecule known to be specifically expressed on the surface of a cancer cell, and wherein the sample is a sample solution containing test cells containing the cancer cell.

11. The method according to claim 8, wherein the microstructure is magnetic, and the method further comprising a step of controlling the orientation of the microstructure by a magnetic field by applying an external magnetic field to the microstructure between step b) and step c).

12. The method according to claim 11, wherein the step of controlling the orientation of the microstructure by the magnetic field comprises arranging the microstructure in an orientation such that the convex surface of the microstructure is in contact with the electrode surface.

13. The method according to claim 8, comprising a step of attaching the convex surface of the microstructure to a cantilever of an atomic electron microscope between step a) and step b).

14. A method for producing a nearly hemispherical shell-shaped microstructure, comprising steps of:
a) preparing nearly hemispherical mold microparticles of a desired size disposed in a monolayer on a substrate, wherein the mold microparticles are made of a material that can be removed by a predetermined removal process;
b) coating the mold microparticles disposed on the substrate in the monolayer with a second conductive material;
c) further coating the mold particles coated with the second conductive material with the first conductive material; and
d) removing the mold microparticles by the predetermined removal process to obtain microstructures having a nearly hemispherical shell-shaped structure made of the first conductive material and an electrode layer made of the second conductive material disposed on the concave side of the nearly hemispherical shell-shaped structure,
wherein the first conductive material comprises a magnetic material,
the second conductive material comprises an electrode material, and
the size (diameter) of the mold microparticle is in the range of about 10 nm to about 50 μm.

15. The method according to claim 14, further comprising the step of further coating the mold particles coated with the second conductive material with the third conductive material between step b) and step c), and in step c), further coating the mold particles coated with the third conductive material with the first conductive material.

16. The method according to claim 14, wherein the step of coating the mold microparticles with the first, second or third conductive material comprises coating the mold microparticles using a thin film deposition device selected from the group consisting of a sputtering device, a resistance heating vacuum deposition device, and a chemical vapor deposition device.

17. The method according to claim 14, wherein the magnetic material comprises nickel, iron, or cobalt.

18. The method according to claim 14, wherein the electrode material comprises nanocarbons, and the thin film formed in the step of coating with the second conductive material comprises a nanocarbon thin film with a mixture of sp²-bonded regions and sp³-bonded regions.

19. The method according to claim 14, wherein the material forming the mold particles comprises a material selected from the group consisting of polystyrene, polypropylene, cellulose, and glass.

20. The method according to claim 14, wherein the predetermined removal process comprises removing the mold particles by a process selected from the group consisting of heating the mold particles to a high temperature, treating the mold particles with an organic solvent, and treating the mold particles with active oxygen.

21. The method according to claim 20, wherein the predetermined removal process comprises heating at a high temperature in an atmosphere with an oxygen concentration of about 15% or less.

22. The method according to claim 14, wherein the cavity surrounded by the electrode layer of the concave surface of the nearly hemispherical shell-shaped structure has a size (diameter) that is capable of receiving at least a single cell.

23. The method according to claim 14, wherein the thickness of each thin film layer formed in the step of coating the mold microparticles with the first, second, or third conductive material is in the range of about 0.1 nm to about 1 mm.
